(19) United States
(12) Patent Application Publication
(10) Pub. No.: US 2024/0036063 A1
(43) Pub. Date: Feb. 1, 2024

(54) BIOMARKER COMPOSITION FOR DIAGNOSING DEGENERATIVE BRAIN DISEASES

(71) Applicant: JHK MEDICAL SCIENCE INC., Daejeon (KR)

(72) Inventor: Seo Yun JO, Daejeon (KR)

(73) Assignee: JHK MEDICAL SCIENCE INC., Daejeon (KR)

(21) Appl. No.: 18/265,254

(22) PCT Filed: Dec. 14, 2020
(86) PCT No.: PCT/KR2020/018263

§ 371 Date: Jun. 4, 2023

(30) Foreign Application Priority Data
Dec. 8, 2020 (KR) 10-2020-0170316

(51) Int. Cl. G01N 33/68; G01N 33/573

(52) U.S. Cl. CPC G01N 33/6896; G01N 33/573; G01N 2333/4709; G01N 2333/988; G01N 2333/99; G01N 2333/948; G01N 2800/28

(57) ABSTRACT

A biomarker composition for diagnosing degenerative brain diseases includes main markers and sub-markers as active ingredients by which, in the blood serum of patients with mild cognitive impairment/stage 1 dementia and stage 2 dementia/stage 3 dementia, the levels of main markers amyloid beta 40 (Aβ40), tau, neuron-specific enolase (NSE), and glial fibrillary acidic protein (GFAP) were measured to be significantly higher than those of a normal control group, the levels of brain-derived nerve growth factor (BDNF) and ubiquitin carboxy-terminal hydrolase L1 (UCH-L1) were measured to be significantly low, the levels of phospho-tau (AT180) and homocystein (HCY) were measured to be significantly high, and the level of peptidyl-prolyl isomerase (Pin1) was confirmed to be significantly low.

### TECHNICAL FIELD

The present disclosure relates to a biomarker composition for diagnosing degenerative brain diseases.

### BACKGROUND ART

Degenerative brain disease refers to a disease that occurs in the brain among degenerative diseases developed with age and may be classified on the basis of the main symptoms and the brain area infiltrated, including, for example, Alzheimer's disease, frontotemporal dementia, lewy body dementia, Parkinson's disease, Huntington's disease, and amyotrophic lateral sclerosis. Degenerative brain diseases are known to occur by neurodegeneration due to aging and the death of nerve cells caused by aggregation of specific proteins for each disease due to genetic and environmental factors, but the exact pathological mechanism has not yet been identified, and basic research to clarify this is being actively conducted.

Alzheimer's disease starts slowly with memory loss and progresses to a gradual, extensive loss of cognitive functions such as loss of visuospatial ability, language impairment, and executive ability impairment, resulting in symptoms of dementia that makes it impossible to carry out independent daily life. Although the pathological mechanism has not yet been precisely identified, it is reported to be derived from an interaction between aging, genetic risk factors, and environmental factors.

According to the results analyzed by dementia-related organizations such as the Ministry of Health and Welfare, the National Health Insurance Service, the Health Insurance Review and Assessment Service, the Social Security Information Service, and the National Statistics Office, the number of dementia patients is estimated to be more than 700,000 at the end of 2017, and the annual management cost per one dementia patient is about 20.74 million won, while the national dementia management cost is about 14.6 trillion won. Currently, the prevention, diagnosis, and management of dementia involves questionnaires such as clinical examination or expensive examinations that requires high price such as PET-CT, and biological samples such as cerebrospinal fluid are required for diagnosis and management after the onset of the disease, increasing the burden on the patients with pain and management costs. Moreover, the number of patients and costs are expected to increase continuously with the aging society, and thus it is a great burden for the country as well, considering the additional loss of social cost thereby.

However, existing technology for diagnosing dementia using blood has been scientifically carried out using a laser analyzer in a unit of pg/ml in serum or plasma, but blood components are not used therefor. Existing related diagnostic kits require simple and insensitive methods with limited markers using the potential difference of dementia markers that lack diversity, such that it is not easy to accurately diagnose and distinguish dementia. Therefore, in order to secure the individual's right to pursue health and reduce the cost of individual/national management, the need for effective dementia diagnostic kits using blood is increasing, so that dementia may be prevented and detected at an early stage and thus the degree of progression may be under control.

### PRIOR ART DOCUMENT

### Patent Document

Korean Patent Application Publication No. 10-2020-0097636 (published on Aug. 19, 2020)

### DISCLOSURE OF THE INVENTION

### Technical Goals

The present disclosure relates to a biomarker composition for diagnosing degenerative brain diseases, and specifically, an object of the present disclosure is to provide a composition including main markers and sub-markers as active ingredients as a biomarker composition for diagnosing degenerative brain diseases, by identifying that levels of main markers amyloid beta 40 (Aβ40), amyloid beta 42 (Aβ42), tau, neuron-specific enolase (NSE), and glial fibrillary acidic protein (GFAP) were measured to be significantly higher than those of a normal control group from the blood serum of patients with mild cognitive impairment/stage 1 dementia and stage 2 dementia/stage 3 dementia, the levels of brain-derived nerve growth factor (BDNF) and ubiquitin carboxy-terminal hydrolase L1 (UCH-L1) were measured to be significantly low, the levels of sub-markers collapsing response mediator protein-2 (CRMP2), N-type voltage-gated calcium channels (CaV2.2), AT180, and homocysteine (HCY) were measured to be significantly high, and the level of peptidyl-prolyl isomerase (Pin1) was significantly low.

### Technical Solutions

The present disclosure provides a composition for diagnosing degenerative brain diseases, including, as active ingredients, one or more main markers selected from the group consisting of amyloid beta 40 (Aβ40), amyloid beta 42 (Aβ42), brain-derived nerve growth factor (BDNF), glial fibrillary acidic protein (GFAP), neuron-specific enolase (NSE), tau, and ubiquitin carboxy-terminal hydrolase L1 (UCH-L1); and one or more sub-markers selected from the group consisting of collapsing response mediator protein-2 (CRMP2), N-type voltage-gated calcium channels (CaV2.2), peptidyl-prolyl isomerase (Pin1), phospho-tau (AT180), and homocysteine (HCY).

In addition, the present disclosure provides a kit for diagnosing degenerative brain diseases, including a preparation for measuring levels of one or more main markers selected from the group consisting of amyloid beta 40 (Aβ40), amyloid beta 42 (Aβ42), brain-derived nerve growth factor (BDNF), glial fibrillary acidic protein (GFAP), neuron-specific enolase (NSE), tau, and ubiquitin carboxy-terminal hydrolase L1 (UCH-L1); and a preparation for measuring levels of one or more sub-markers selected from the group consisting of collapsing response mediator protein-2 (CRMP2), N-type voltage-gated calcium channels (CaV2.2), peptidyl-prolyl isomerase (Pin1), phospho-tau (AT180), and homocysteine (HCY).

In addition, the present disclosure provides a method of providing information for diagnosing degenerative brain diseases, including measuring levels of one or more main markers selected from the group consisting of amyloid beta 40 (Aβ40), amyloid beta 42 (Aβ42), brain-derived nerve growth factor (BDNF), glial fibrillary acidic protein (GFAP), neuron-specific enolase (NSE), tau, and ubiquitin carboxy-terminal hydrolase L1 (UCH-L1) from a biological sample isolated from a subject; comparing the levels of the main markers with a normal control group; measuring levels of one or more sub-markers selected from the group consisting of collapsing response mediator protein-2 (CRMP2), N-type voltage-gated calcium channels (CaV2.2), peptidyl-prolyl isomerase (Pin1), phospho-tau (AT180), and homocysteine (HCY) from the biological sample; and comparing the levels of the sub-markers with the normal control group.

### Advantageous Effects

According to the present disclosure, it is possible to provide a composition including main markers and sub-markers as active ingredients as a biomarker composition for diagnosing degenerative brain diseases, by identifying that levels of main markers amyloid beta 40 (Aβ40), tau, neuron-specific enolase (NSE), and glial fibrillary acidic protein (GFAP) were measured to be significantly higher than those of a normal control group from the blood serum of patients with mild cognitive impairment/stage 1 dementia and stage 2 dementia/stage 3 dementia, the levels of brain-derived nerve growth factor (BDNF) and ubiquitin carboxy-terminal hydrolase L1 (UCH-L1) were measured to be significantly low, the levels of collapsing response mediator protein-2 (CRMP2), N-type voltage-gated calcium channels (CaV2.2), phospho-tau (AT180), and homocysteine (HCY) were significantly high, and the level of peptidyl-prolyl isomerase (Pin1) was significantly low.

### BRIEF DESCRIPTION OF DRAWINGS

FIG. 1 shows a schematic diagram illustrating a method of measuring levels of main markers or sub-markers, which are biomarkers for diagnosing degenerative brain diseases.

FIG. 2 shows a result of evaluating amyloid beta 40 (Aβ40), a biomarker for diagnosing degenerative brain diseases.

FIG. 3 shows a result of evaluating amyloid beta 42 (Aβ42), a biomarker for diagnosing degenerative brain diseases.

FIG. 4 shows a result of evaluating brain-derived nerve growth factor (BDNF), a biomarker for diagnosing degenerative brain diseases.

FIG. 5 shows a result of evaluating glial fibrillary acidic protein (GFAP), a biomarker for diagnosing degenerative brain diseases.

FIG. 6 shows a result of evaluating neuron-specific enolase (NSE), a biomarker for diagnosing degenerative brain diseases.

FIG. 7 shows a result of evaluating tau, a biomarker for diagnosing degenerative brain diseases.

FIG. 8 shows a result of evaluating ubiquitin carboxy-terminal hydrolase L1 (UCH-L1), a biomarker for diagnosing degenerative brain diseases.

FIG. 9 shows a result of evaluating collapsing response mediator protein-2 (CRMP2), a biomarker for diagnosing degenerative brain diseases.

FIG. 10 shows a result of evaluating N-type voltage-gated calcium channel (CaV 2.2), a biomarker for diagnosing degenerative brain diseases.

FIG. 11 shows a result of evaluating peptidyl-prolyl isomerase (Pin1), a biomarker for diagnosing degenerative brain diseases.

FIG. 12 shows a result of evaluating phospho-tau (AT180), a biomarker for diagnosing degenerative brain diseases.

FIG. 13 shows a result of evaluating homocysteine (HCY), a biomarker for diagnosing degenerative brain diseases.

### BEST MODE FOR CARRYING OUT THE INVENTION

The terms used herein have been selected from currently widely used general terms as much as possible in consideration of functions herein, but these may vary depending on the intentions or precedents of those skilled in the art, the emergence of new technologies, and the like. In addition, in specific cases, there are terms arbitrarily selected by the applicant, and in this case, the meaning will be described in detail in the description of the disclosure. Therefore, the terms used herein should not be defined as simple names of terms, but based on the meaning of the term and the overall contents of the present disclosure.

Unless otherwise defined, all terms used herein, including technical or scientific terms, have the same meaning as commonly understood by those skilled in the art to which the present disclosure pertains. Terms such as those defined in commonly used dictionaries should be construed as having meanings consistent with the meaning in the context of the relevant art and are not to be construed in an ideal or overly formal meaning unless clearly defined in the present application.

The numerical range includes the numerical value defined in the above range. All maximum numerical limits given herein include all lower numerical limits as clearly stated on the lower numerical limits. All minimum numerical limits given herein include all higher numerical limits as clearly stated on the higher numerical limits. All numerical limits given herein will include all better numerical ranges within a wider numerical range as clearly stated on narrower numerical limits.

Hereinafter, the present disclosure will be described in more detail.

The present disclosure provides a composition for diagnosing a degenerative brain disease, including, as active ingredients, one or more main markers selected from the group consisting of amyloid beta 40 (Aβ40), amyloid beta 42 (Aβ42), brain-derived nerve growth factor (BDNF), glial fibrillary acidic protein (GFAP), neuron-specific enolase (NSE), tau, and ubiquitin carboxy-terminal hydrolase L1 (UCH-L1); and one or more sub-markers selected from the group consisting of collapsing response mediator protein-2 (CRMP2), N-type voltage-gated calcium channels (CaV2.2), peptidyl-prolyl isomerase (Pin1), phospho-tau (AT180), and homocysteine (HCY).

The degenerative brain disease may be any one selected from the group consisting of Alzheimer's disease, frontotemporal dementia, Parkinson's disease, amyotrophic lateral sclerosis, progressive supranuclear palsy, corticobasal degeneration, Pick's disease, mild cognitive impairment (MCI), and dementia.

In addition, the present disclosure provides a kit for diagnosing a degenerative brain disease, including a preparation for measuring levels of one or more main markers selected from the group consisting of amyloid beta 40 (Aβ40), amyloid beta 42 (Aβ42), brain-derived nerve growth factor (BDNF), glial fibrillary acidic protein (GFAP), neuron-specific enolase (NSE), tau, and ubiquitin carboxy-terminal hydrolase L1 (UCH-L1); and a preparation for measuring levels of one or more sub-markers selected from the group consisting of collapsing response mediator protein-2 (CRMP2), N-type voltage-gated calcium channels (CaV2.2), peptidyl-prolyl isomerase (Pin1), phospho-tau (AT180), and homocysteine (HCY).

The preparation for measuring the levels of the main markers is an antibody that specifically binds to the main markers, and the preparation for measuring the levels of the sub-markers is an antibody that specifically binds to the sub-markers. The antibodies are commercially available.

The kit may measure the levels of a main marker and a sub-marker present in plasma in a concentration range of 0.3 pg/ml to 50 ng/ml.

In addition, the present disclosure provides a method of providing information for diagnosing a degenerative brain disease, including measuring levels of one or more main markers selected from the group consisting of amyloid beta 40 (Aβ40), amyloid beta 42 (Aβ42), brain-derived nerve growth factor (BDNF), glial fibrillary acidic protein (GFAP), neuron-specific enolase (NSE), tau, and ubiquitin carboxy-terminal hydrolase L1 (UCH-L1) from a biological sample isolated from a subject; comparing the levels of the main markers with a normal control group; measuring levels of one or more sub-markers selected from the group consisting of collapsing response mediator protein-2 (CRMP2), N-type voltage-gated calcium channels (CaV2.2), peptidyl-prolyl isomerase (Pin1), phospho-tau (AT180), and homocysteine (HCY) from the biological sample; and comparing the levels of the sub-markers with a normal control group.

The measuring of the levels of the main markers includes treating the biological sample with a primary antibody that specifically binds to amyloid beta 40 (Aβ40), amyloid beta 42 (Aβ42), brain-derived nerve growth factor (BDNF), glial fibrillary acidic protein (GFAP), neuron-specific enolase (NSE), tau, or ubiquitin carboxy-terminal hydrolase L1 (UCH-L1); treating a secondary antibody that specifically binds to the primary antibody and is in a form in which biotin is conjugated to a constant region; and treating avidin that specifically binds to the biotin and is in a form having horseradish peroxidase (HRP) conjugated.

The measuring of the levels of the sub-markers includes treating the biological sample with a primary antibody that specifically binds to collapsing response mediator protein-2 (CRMP2), N-type voltage-gated calcium channels (CaV2.2), peptidyl-prolyl isomerase (Pin1), phospho-tau (AT180), and homocysteine (HCY); treating a secondary antibody that specifically binds to the primary antibody and is in a form in which biotin is conjugated to a constant region; and treating avidin that specifically binds to the biotin and is in a form having horseradish peroxidase (HRP) conjugated.

The method of providing information further includes determining, if the levels of amyloid beta 40 (Aβ40), tau, neuron-specific enolase (NSE), and glial fibrillary acidic protein (GFAP) are higher than those of the normal control group and the levels of amyloid beta 42 (Aβ42), brain-derived nerve growth factor (BDNF), and ubiquitin carboxy-terminal hydrolase L1 (UCH-L1) are lower than those of the normal control group, that the subject is to diagnosed with or likely to be diagnosed with a degenerative brain disease.

The method of providing information further includes determining, if the levels of collapsing response mediator protein-2 (CRMP2), N-type voltage-gated calcium channels (CaV2.2), phospho-tau (AT180), and homocysteine (HCY) are high and the level of peptidyl-prolyl isomerase (Pin1) is lower than that of the normal, that the subject is diagnosed with or likely to be diagnosed with a degenerative brain disease.

### MODES FOR CARRYING OUT THE INVENTION

Hereinafter, Experimental Examples and Examples will be described in detail to help the understanding of the present disclosure. However, the following Experimental Examples and Examples are merely illustrative of the content of the present disclosure, and the scope of the present disclosure is not limited to the following Experimental Examples and Examples. The Experimental Examples and Examples of the present disclosure are provided to more completely explain the present disclosure to those skilled in the art.

### Example

In accordance with the present disclosure, indirect immunometric ELISA developed by the present inventors was used to measure the levels of main markers and sub-markers for diagnosing degenerative brain diseases.

Used to measure the levels of main markers or sub-markers were antibodies that specifically bind to amyloid beta 40 (Aβ40), amyloid beta 42 (Aβ42), brain-derived nerve growth factor (BDNF), glial fibrillary acidic protein (GFAP), neuron-specific enolase (NSE), tau, ubiquitin carboxy-terminal hydrolase L1 (UCH-L1), collapsing response mediator protein-2 (CRMP2), N-type voltage-gated calcium channels (CaV2.2), peptidyl-prolyl isomerase (Pin1), phospho-tau (AT180), or homocysteine (HCY). An antibody having biotin conjugated was used.

The antibodies and experimental methods used to measure the level of each biomarker were based on commercially available kits. Specifically, Human Amyloid beta 40 ELISA Kit (Invitrogen) was used for amyloid beta 40 (Aβ40), Human Amyloid beta 42 ELISA Kit, Ultrasensitive (Invitrogen) for amyloid beta 42 (Aβ42), Human Brain-derived neurotrophic factor ELISA Kit (abcam) for brain-derived nerve growth factor (BDNF), Human Glial fibrillary acidic protein DuoSet ELISA (R&D Systems) for glial fibrillary acidic protein (GFAP), Human Neuron-specific Enolase ELISA Kit (abcam) for neuron-specific enolase (NSE), Human Tau ELISA Kit (abcam) for tau, Human Ubiquitin carboxy-terminal hydrolase L1 DuoSet ELISA (R&D Systems) for ubiquitin carboxy-terminal hydrolase L1 (UCH-L1), Human DPYSL2/CRMP2 ELISA Kit (LSBio) for collapsing response mediator protein-2 (CRMP2), Human Voltage dependent N-type calcium channels subunit Alpha 1B ELISA kit (Mybiosource) for N-type voltage-gated calcium channels (CaV2.2), Human peptidylprolyl cis/trans isomerase NIMA-interacting) ELISA Kit (Cusabio) for peptidyl-prolyl isomerase (Pin1), Human Phosphorylated tau 231 ELISA Kit (Mybiosource) for phospho-tau (AT180), and Human Homocysteine ELISA Kit (Mybiosource) for homocysteine (HCY). The materials used in Example are as shown in Table 1 below.

TABLE 1

| Reagents | Quantity | |
|---|---|---|
| Assay plate (12 × 8 coated microwells) | 1(96 wells) | |
| Standard (Freeze dried) | 2 | |
| Biotin-antibody (100 × concentrate) | 1 × 120 | μl |
| HRP-avidin (100 × concentrate) | 1 × 120 | μl |
| Biotin-antibody Diluent | 1 × 15 | ml |
| HRP-avidin Diluent | 1 × 15 | ml |
| Sample Diluent | 1 × 50 | ml |
| Wash Buffer (25 × concentrate) | 1 × 20 | ml |
| TMB Substrate | 1 × 10 | ml |
| Stop Solution | 1 × 10 | ml |

| | |
|---|---|
| Adhesive Strip (For 96 wells) | 4 |
| Instruction manual | 1 |

The biotin-antibody is an antibody having biotin conjugated and specifically binds to a biomarker. The antibody was used by being diluted 100 times in a biotin-antibody diluent solution. The HRP-avidin is an avidin substance that binds to biotin conjugated to an antibody and has a form having horseradish peroxidase (HRP) conjugated, which was used by being diluted 100 times in HRP-avidin diluent solution. TMB Substrate Set [BioLegend, 421101] was used for TMB substrate, and 1 M sulfuric acid (H2SO4) [PFP, 8J250] was used for the stop solution. 20 ml of wash buffer (25×) was diluted in deionized water or distilled water to prepare 500 ml. A stock solution for standard of each of the main marker and sub-marker was prepared and diluted by ½ to prepare a standard sample at a concentration of 0 pg/ml to 50 ng/ml. 10× anti-Rabbit IgG HRP was used as HRP diluent, and 1× anti-Rabbit IgG HRP was used.

A sample to be analyzed was prepared to measure the level of the main marker or sub-marker. Prepared were samples that had been stored at 2-8° C. within 5 days from the day of collection. The prepared sample was coagulated for 2 hours using a serum separator tube (SST) and then centrifuged at 1000×g for 15 min. Plasma EDTA or heparin was used as an anticoagulant to collect plasma. Within 30 minutes after collection, centrifugation was performed at 2-8° C. for 15 minutes under conditions of 1000×g. Cell cultures were used by undergoing centrifugation at 1000×g and 2-8° C. for 15 minutes to remove particulates. Tissue homogenates were used after rinsing 100 mg of tissue with 1×PBS, homogenizing in 1 mL of 1×PBS, undergoing two freeze-thaw cycles, and then centrifuging at 5000×g and 2-8° C. for minutes to remove the supernatant.

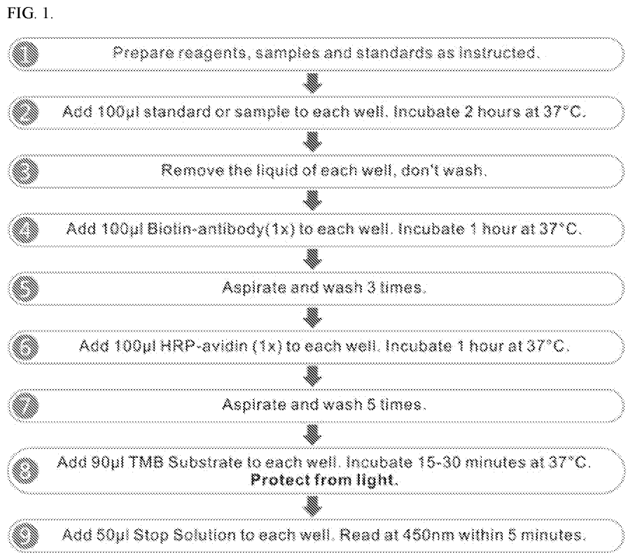

As shown in FIG. 1, each serum sample or standard sample was added to the well by 100 μl, and the well was covered with an adhesive strip, followed by culture at 37° C. for 2 hours. 100 μl of biotin-antibody (1×) was added to each well, and replacement took place with a new adhesive strip, followed by culture at 37° C. for 1 hour. Each well was washed 3 times using 200 μl of wash buffer. 100 μl of HRP-avidin (1×) was added, and replacement took place with a new adhesive strip, followed by culture at 37° C. for 1 hour. Each well was washed 3 times using 200 μl of wash buffer. 90 μl of TMB substrate was added to each well which was then cultured at 37° C. for 15 to 30 minutes under dark conditions. 50 μl of stop solution was added to stop the reaction. A microplate reader was used to measure the absorbance at 450 nm.

Based on absorbance values changing from colorless or light blue to blue gradient, the levels of the main markers or sub-markers were calculated. A standard curve was created by a “Curve Expert” using the result values of the standard sample, and a standard curve was generated by reducing data using computer software that may generate a four-parameter logistic (4 PL) curve fitting. The optimal curve was measured by points on the graph on the x-axis for the concentration of the y-axis.

### Test Example

In order to identify the main markers and sub-markers for diagnosing degenerative brain diseases according to the present disclosure, blood samples were provided from normal volunteers, patients with mild cognitive impairment, patients with stage 1 dementia, patients with stage 2 dementia, and patients with stage 3 dementia, and the levels of main markers and sub-markers present in each serum were measured. Samples of volunteers recruited from Aug. 5, 2020 to Oct. 31, 2020 at Chuncheon Saeyun Nursing Hospital in Gangwon Province were used, and written consent was given to provide information.

Samples received from 10 normal volunteers were classified as normal control groups, those received from 23 patients with mild cognitive impairment and stage 1 dementia were classified into the first experimental group (mild cognitive impairment/stage 1 dementia), and those received from 22 patients with stage 2 dementia and stage 3 dementia were classified into the second experimental group (stage 2 dementia/stage 3 dementia). Using the method of the above Example, the levels of amyloid beta 40 (Aβ40), amyloid beta 42 (Aβ42), brain-derived nerve growth factor (BDNF), glial fibrillary acidic protein (GFAP), neuron-specific enolase (NSE), tau, ubiquitin carboxy-terminal hydrolase L1 (UCH-L1), collapsing response mediator protein-2 (CRMP2), N-type voltage-gated calcium channels (CaV2.2), peptidyl-prolyl isomerase (Pin1), phospho-tau (AT180), or homocysteine (HCY) were measured from blood samples of each classified control group and experimental group

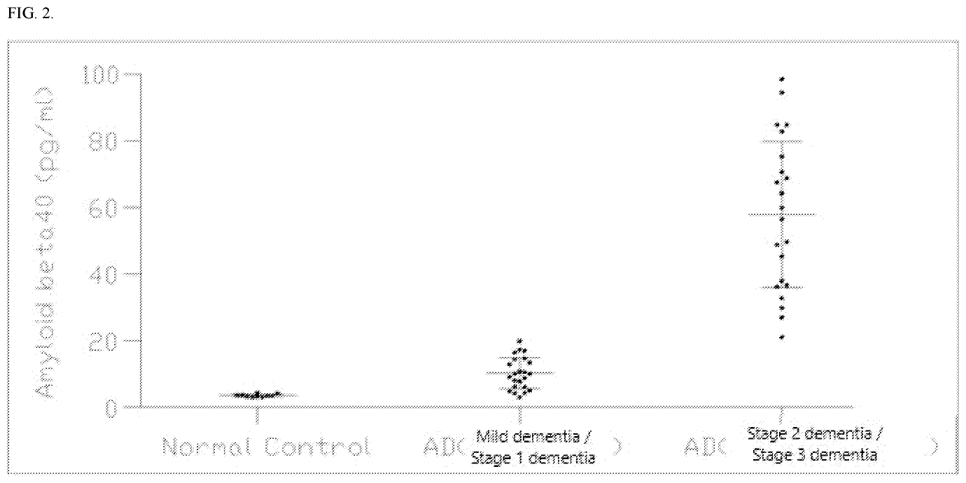

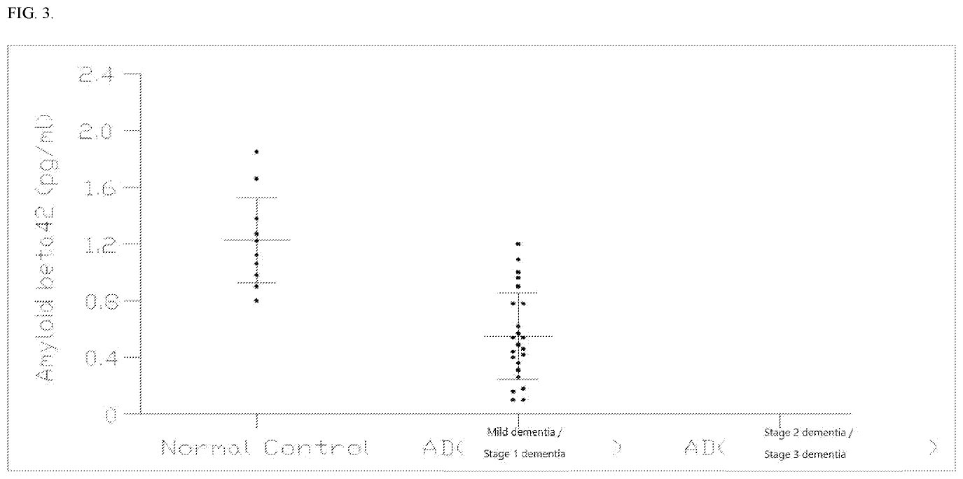

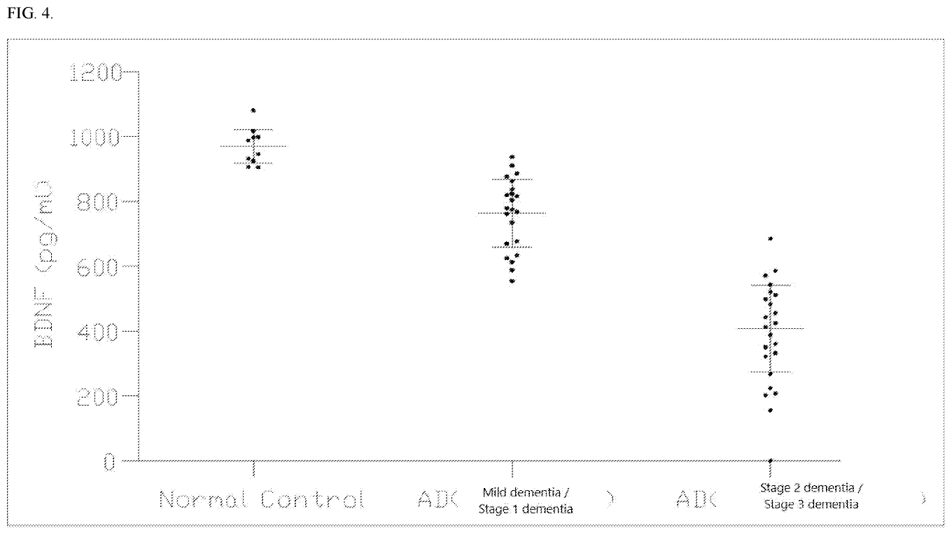

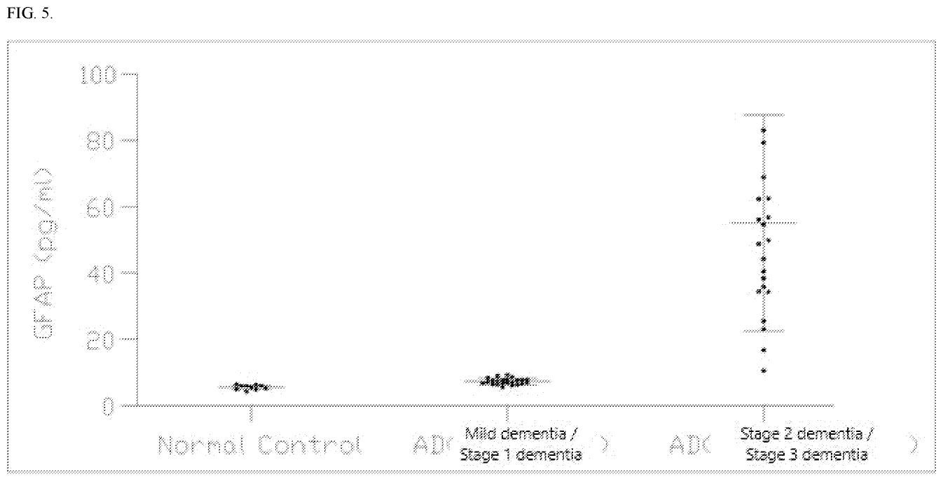

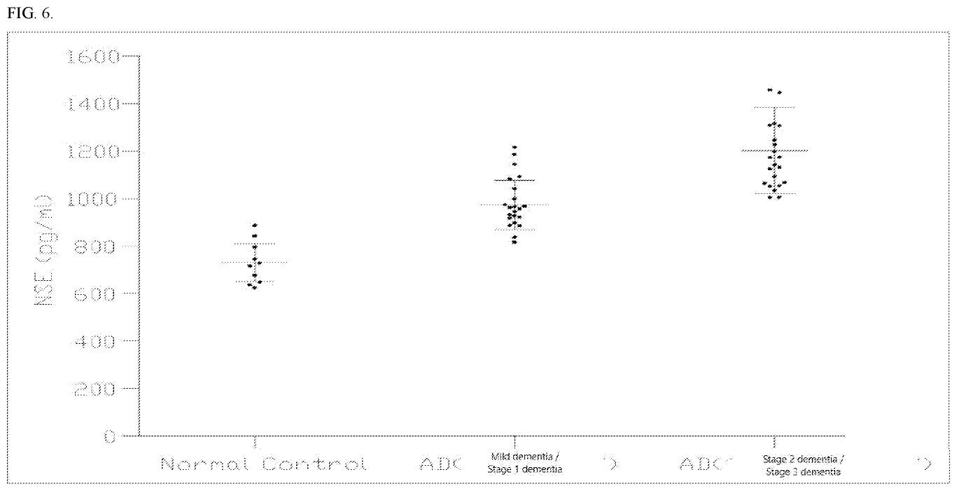

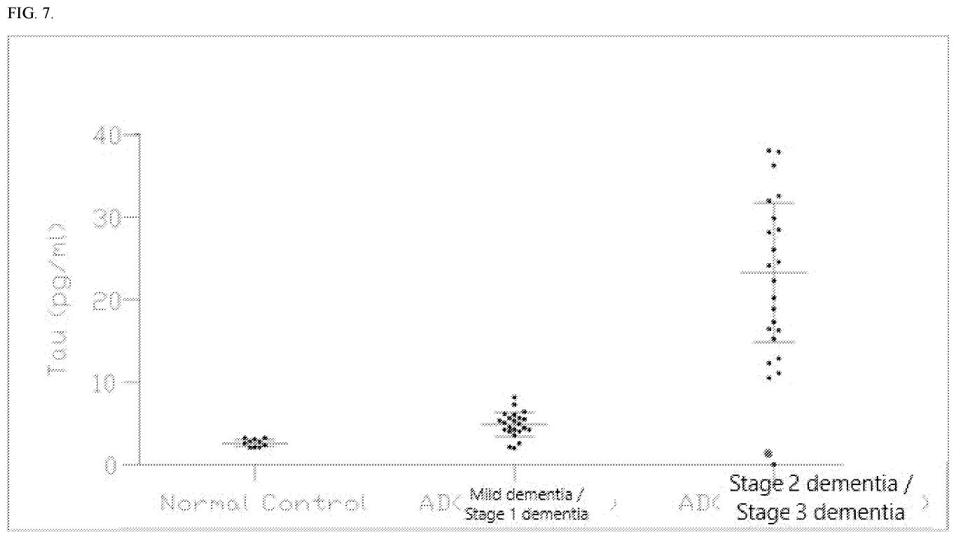

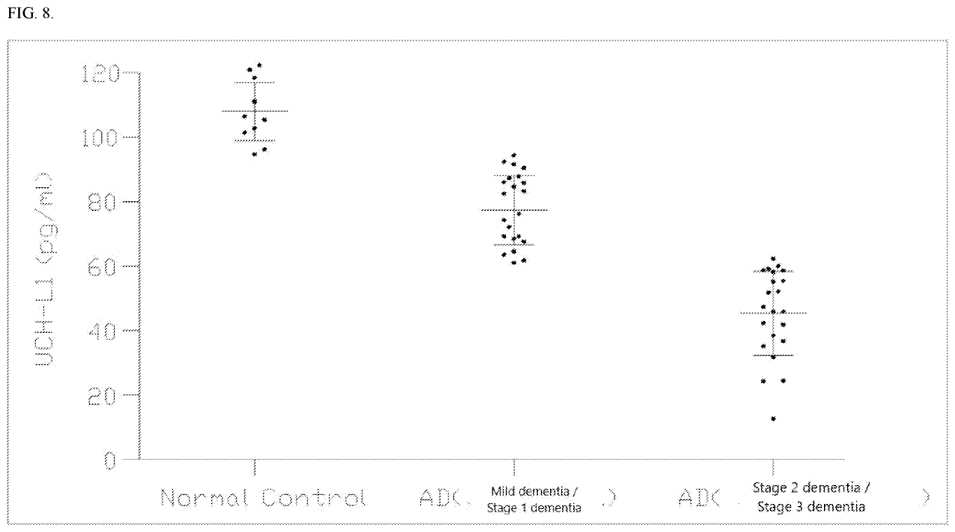

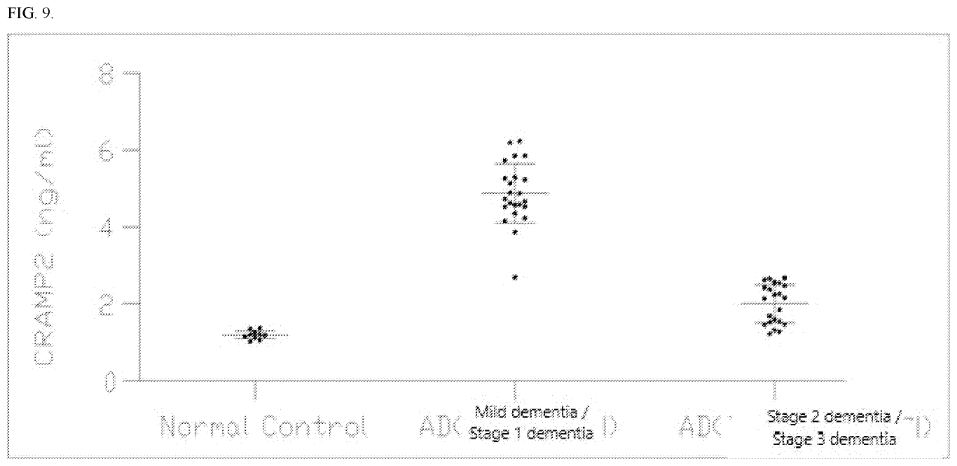

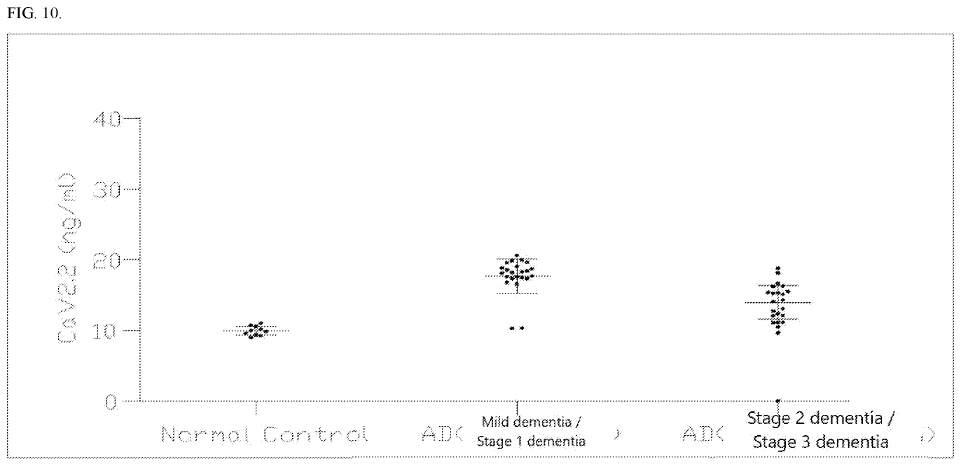

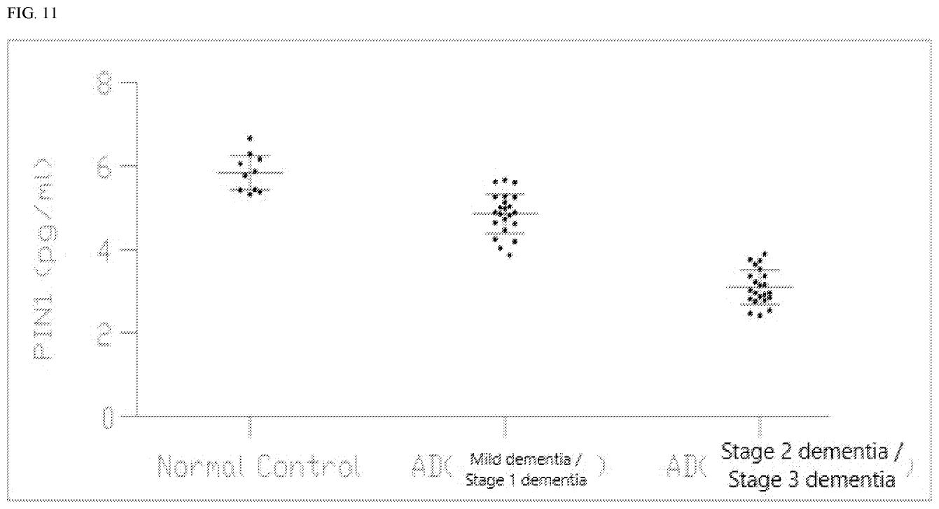

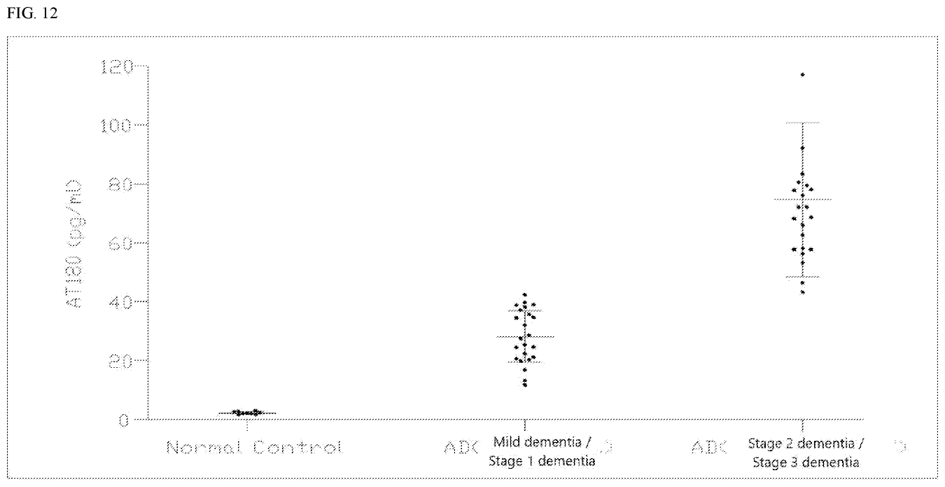

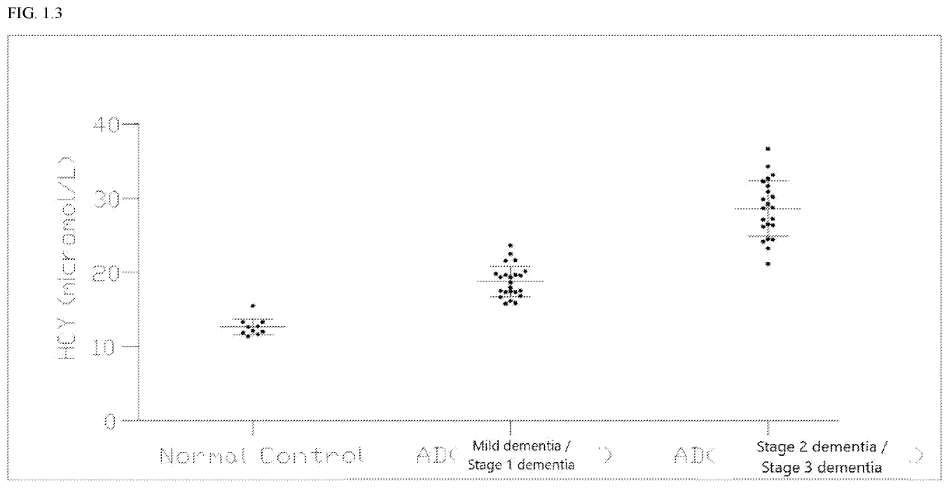

As shown in FIGS. 2 to 13 above, it was observed that the levels of the main markers amyloid beta 40 (Aβ40), tau, neuron-specific enolase (NSE), and glial fibrillary acidic protein (GFAP) were significantly high in the first experimental group (mild cognitive impairment/stage 1 dementia) and the second experimental group (stage 2 dementia/stage 3 dementia) compared to the normal control group. It was observed that the levels of the main markers amyloid beta 42 (Aβ42), brain-derived nerve growth factor (BDNF), and ubiquitin carboxy-terminal hydrolase L1 (UCH-L1) were significantly low in the first experimental group (mild cognitive impairment/stage 1 dementia) and the second experimental group (stage 2 dementia/stage 3 dementia) compared to the normal control group.

In addition, the levels of sub-markers collapsing response mediator protein-2 (CRMP2) and N-type voltage-gated calcium channels (CaV2.2) were significantly high in the first experimental group (mild cognitive impairment/stage 1 dementia) but those in the second experimental group (stage 2 dementia/stage 3 dementia) were significantly low compared to the normal control group. The levels of sub-markers phospho-tau (AT180) and homocysteine were significantly high in the first experimental group (mild cognitive impairment/stage 1 dementia) and the second experimental group (stage 2 dementia/stage 3 dementia) compared to the normal control group. The level of a sub-marker peptidyl-prolyl isomerase (Pin1) was significantly low in the first experimental group (mild cognitive impairment/stage 1 dementia) and the second experimental group (stage 2 dementia/stage 3 dementia) compared to the normal control group.

As described above, a specific part of the content of the present disclosure is described in detail, for those of ordinary skill in the art, it is clear that the specific description is only a preferred example embodiment, and the scope of the present disclosure is not limited thereby. In other words, the substantial scope of the present disclosure may be defined by the appended claims and their equivalents.

1. A composition for diagnosing a degenerative brain disease, comprising, as active ingredients:
one or more main markers selected from the group consisting of amyloid beta 40 (Aβ40), amyloid beta 42 (Aβ42), brain-derived nerve growth factor (BDNF), glial fibrillary acidic protein (GFAP), neuron-specific enolase (NSE), tau, and ubiquitin carboxy-terminal hydrolase L1 (UCH-L1); and
one or more sub-markers selected from the group consisting of collapsing response mediator protein-2 (CRMP2), N-type voltage-gated calcium channels (CaV2.2), peptidyl-prolyl isomerase (Pin1), phospho-tau (AT180), and homocysteine (HCY).

2. The composition of claim 1, wherein the degenerative brain disease is any one or more selected from the group consisting of Alzheimer's disease, frontotemporal dementia, Parkinson's disease, amyotrophic lateral sclerosis, progressive supranuclear palsy, corticobasal degeneration, Pick's disease, mild cognitive impairment (MCI), and dementia.

3. A kit for diagnosing a degenerative brain disease, comprising:
a preparation for measuring levels of one or more main markers selected from the group consisting of amyloid beta 40 (Aβ40), amyloid beta 42 (Aβ42), brain-derived nerve growth factor (BDNF), glial fibrillary acidic protein (GFAP), neuron-specific enolase (NSE), tau, and ubiquitin carboxy-terminal hydrolase L1 (UCH-L1); and
a preparation for measuring levels of one or more sub-markers selected from the group consisting of collapsing response mediator protein-2 (CRMP2), N-type voltage-gated calcium channels (CaV2.2), peptidyl-prolyl isomerase (Pin1), phospho-tau (AT180), and homocysteine (HCY).

4. The kit of claim 3, wherein the preparation for measuring the levels of the main markers is an antibody that specifically binds to the main markers, and the preparation for measuring the levels of the sub-markers is an antibody that specifically binds to the sub-markers.

5. The kit of claim 3, wherein the kit measures the levels of a main marker and a sub-marker present in plasma in a concentration range of 0.3 pg/ml to 50 ng/ml.

6. A method of providing information for diagnosing a degenerative brain disease, comprising:
measuring levels of one or more main markers selected from the group consisting of amyloid beta 40 (Aβ40), amyloid beta 42 (Aβ42), brain-derived nerve growth factor (BDNF), glial fibrillary acidic protein (GFAP), neuron-specific enolase (NSE), tau, and ubiquitin carboxy-terminal hydrolase L1 (UCH-L1) from a biological sample isolated from a subject;
comparing the levels of the main markers with a normal control group;
measuring levels of one or more sub-markers selected from the group consisting of collapsing response mediator protein-2 (CRMP2), N-type voltage-gated calcium channels (CaV2.2), peptidyl-prolyl isomerase (Pin1), phospho-tau (AT180), and homocysteine (HCY) from the biological sample; and
comparing the levels of the sub-markers with a normal control group.

7. The method of claim 6, wherein the biological sample further comprises one or more components selected from the group consisting of blood, serum, tissue, urine, saliva, and cerebrospinal fluid.

8. The method of claim 6, wherein the measuring of the levels of the main markers comprises:
treating the biological sample with a primary antibody that specifically binds to amyloid beta 40 (Aβ40), amyloid beta 42 (Aβ42), brain-derived nerve growth factor (BDNF), glial fibrillary acidic protein (GFAP), neuron-specific enolase (NSE), tau, or ubiquitin carboxy-terminal hydrolase L1 (UCH-L1);
treating a secondary antibody that specifically binds to the primary antibody and is in a form in which biotin is conjugated to a constant region; and
treating avidin that specifically binds to the biotin and is in a form having horseradish peroxidase (HRP) conjugated.

9. The method of claim 6, wherein the measuring of the levels of the sub-markers comprises:
treating the biological sample with a primary antibody that specifically binds to collapsing response mediator protein-2 (CRMP2), N-type voltage-gated calcium channels (CaV2.2), peptidyl-prolyl isomerase (Pin1), phospho-tau (AT180), and homocysteine (HCY);
treating a secondary antibody that specifically binds to the primary antibody and is in a form in which biotin is conjugated to a constant region; and
treating avidin that specifically binds to the biotin and is in a form having horseradish peroxidase (HRP) conjugated.

10. The method of claim 6, further comprising: determining, if the levels of amyloid beta 40 (Aβ40), tau, neuron-specific enolase (NSE), and glial fibrillary acidic protein (GFAP) are higher than those of the normal control group and the levels of amyloid beta 42 (Aβ42), brain-derived nerve growth factor (BDNF), and ubiquitin carboxy-terminal hydrolase L1 (UCH-L1) are lower than those of the normal control group, that the subject is diagnosed with or likely to be diagnosed with a degenerative brain disease.

11. The method of claim 6, further comprising: determining, if the levels of collapsing response mediator protein-2 (CRMP2), N-type voltage-gated calcium channels (CaV2.2), phospho-tau (AT180), and homocysteine (HCY) are higher than those of the normal control group and the level of peptidyl-prolyl isomerase (Pin1) is lower than that of the normal control group, that the subject is diagnosed with or likely to be diagnosed with a degenerative brain disease.

12. The method of claim 6, wherein the degenerative brain disease is any one or more selected from the group consisting of Alzheimer's disease, frontotemporal dementia, Parkinson's disease, amyotrophic lateral sclerosis, progressive supranuclear palsy, corticobasal degeneration, Pick's disease, mild cognitive impairment (MCI), and dementia.
